(19) United States
(12) Patent Application Publication
(10) Pub. No.: US 2016/0352135 A1
(43) Pub. Date: Dec. 1, 2016

(54) ELECTRONIC BRACELET

(71) Applicant: HON HAI PRECISION INDUSTRY CO., LTD., New Taipei (TW)

(72) Inventors: TZU-SHENG KO, New Taipei (TW); HUI-YU CHAN, New Taipei (TW); JYUN-MENG YANG, New Taipei (TW); PO-CHING CHIANG, New Taipei (TW); KENG-CHIH LIN, New Taipei (TW)

(21) Appl. No.: 14/755,491

(22) Filed: Jun. 30, 2015

(30) Foreign Application Priority Data
May 29, 2015 (CN) 201510285400.6

(51) Int. Cl. H02J 7/02; A61B 5/00; A61B 5/024; H02J 7/04; A61B 5/021

(52) U.S. Cl. CPC H02J 7/025; H02J 7/027; H02J 7/045; A61B 5/021; A61B 5/02438; A61B 5/6824; A61B 5/742; A61B 2560/0214

(57) ABSTRACT

An electronic bracelet includes a rechargeable battery, a chip and a wireless charging module. The chip detects health information of a wearer of the electronic bracelet. The wireless charging module receives an alternating magnetic field and converts the alternating magnetic field to direct current to charge the rechargeable battery.

### CROSS-REFERENCE TO RELATED APPLICATIONS

This application claims priority to Chinese Patent Application No. 201510285400.6, filed on May 29, 2015 the contents of which are incorporated by reference herein.

### FIELD

The subject matter herein generally relates to an electronic bracelet.

### BACKGROUND

Electronic bracelets have functions of obtaining blood pressure and heart of people who wear the electronic bracelet. The functions of the electronic bracelet are generally turned on. Thus, the power of a battery in the electronic bracelet may be quickly run out. The battery is charged through a cable connected the electronic bracelet and a power socket.

### BRIEF DESCRIPTION OF THE DRAWINGS

Implementations of the present technology will now be described, by way of example only, with reference to the attached figures.

FIG. 1 is an isometric view of an electronic bracelet.

FIG. 2 is a block diagram of the electronic bracelet of FIG. 1, according to an embodiment.

FIG. 3 is a block diagram of the electronic bracelet of FIG. 1, according to another embodiment.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

The term “comprising” means “including, but not necessarily limited to”; it specifically indicates open-ended inclusion or membership in a so-described combination, group, series, and the like.

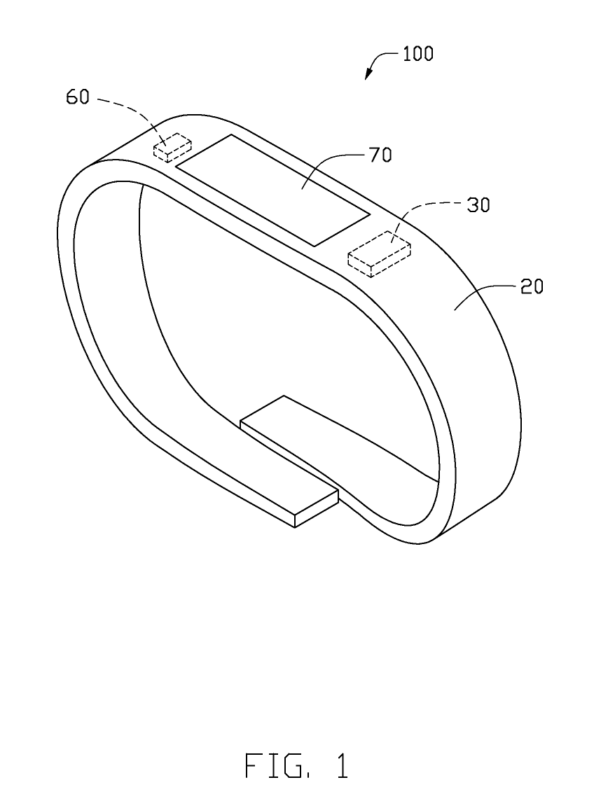

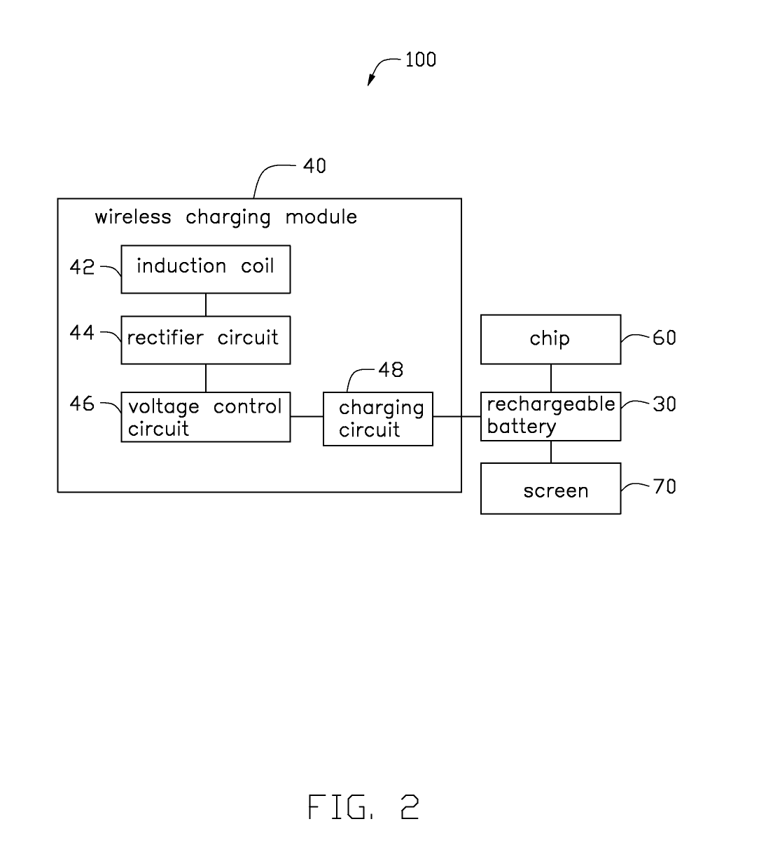

FIGS. 1 and 2 illustrate that an electronic bracelet 100 includes a base 20, a rechargeable battery 30, a wireless charging module 40, a chip 60 and a screen 70. The base 20 is worn on a user. The wireless charging module 40, the chip 60 and the screen 70 are attached in the base. The screen 70 is exposed out of the base 20. The wireless charging module 40 is configured to receive an alternating magnetic field and convert the alternating magnetic field to direct current to charge the rechargeable battery 30. The rechargeable battery 30 provides power to the chip 60 and the screen 70.

The wireless charging module 40 includes an induction coil 42, a rectifier circuit 44, a voltage control circuit 46 and a charging circuit 48. The induction coil 42 is configured to sense the alternating magnetic field and generate inductive oscillating current to provide power to the rectifier circuit 44. The rectifier circuit 44 rectifies the current through the rectifier circuit 44. The voltage control circuit 46 connects to the rectifier circuit 44. The voltage control circuit 46 filters spike voltage of the current through the voltage control circuit 46 and outputs a 5V direct current. The charging circuit 48 connects the voltage control circuit 46. The charging circuit 48 transfers the 5V direct current to the rechargeable battery 30 to charge the rechargeable battery 30.

The chip 60 is configured to detect health information of people who wear the electronic bracelet 100. The health information includes blood pressure and heart of the people. The screen 70 connects to the chip 60. The screen 70 display the obtained health information of the people.

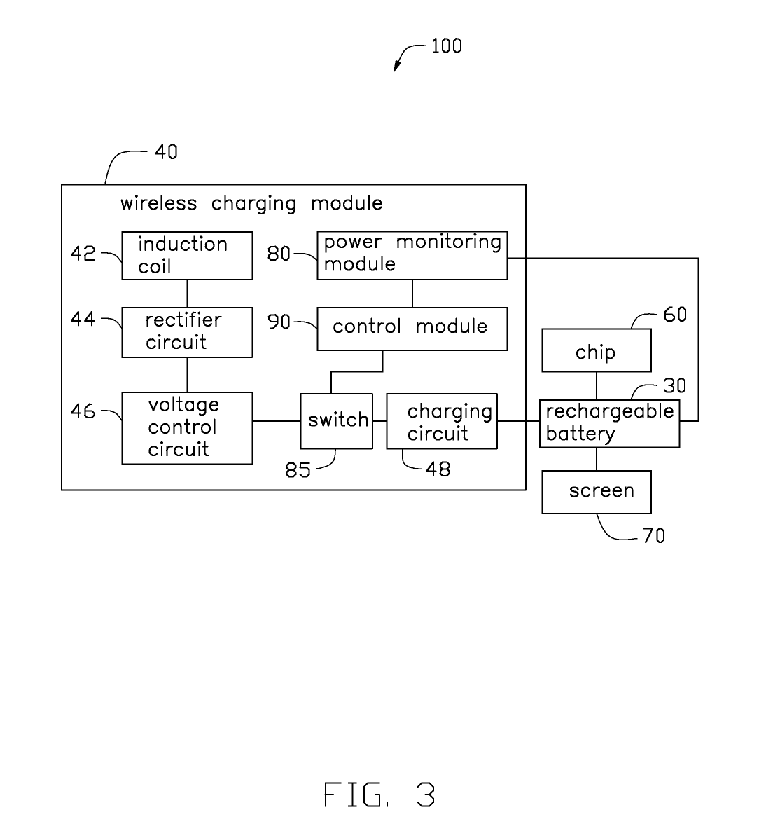

FIG. 3 illustrates that the electronic bracelet 100 further includes a power monitoring module 80, a switch 85 and a control module 90. The power monitoring module 80 is configured to monitor a level of the rechargeable battery 30. The switch 85 is positioned between the wireless charging module 40 and the rechargeable battery 30 and controls whether the wireless charging module 40 connects to the rechargeable battery 30. The control module 90 connects the power monitoring module 80 and the switch 85. The control module 90 is configured to control the switch 85 to be turned on to connect the wireless charging module 40 to the rechargeable battery 30 when the level of the rechargeable battery 30 is less than a first preset value, and control the switch 85 to be turned off to disconnect the wireless charging module 40 to the rechargeable battery 30 when the level of the rechargeable battery 30 is more than a second preset value. Thus, when the level of the rechargeable battery 30 is less than the first preset value, the wireless charging module 40 can charge the rechargeable battery 30 if the wireless charging module 40 receives an alternating magnetic field. When the level of the rechargeable battery 30 is more than the second preset value, the wireless charging module 40 stops charging the rechargeable battery 30. The screen 70 further connects to the power monitoring module 80. The screen 70 further displays the level of the rechargeable battery 30.

The embodiments shown and described above are only examples. Even though numerous characteristics and advantages of the present technology have been set forth in the foregoing description, together with details of the structure and function of the present disclosure, the disclosure is illustrative only, and changes may be made in the details, including in matters of shape, size, and arrangement of the parts within the principles of the present disclosure, up to and including the full extent established by the broad general meaning of the terms used in the claims.

What is claimed is:

1. An electronic bracelet comprising:
a rechargeable battery;
a chip configured to detect health information of a wearer of the electronic bracelet; and
a wireless charging module configured to receive an alternating magnetic field and convert the alternating magnetic field to direct current to charge the rechargeable battery.

2. The electronic bracelet as claimed in claim 1, wherein the wireless charging module comprises an induction coil, the induction coil senses the alternating magnetic field and generate inductive oscillating current.

3. The electronic bracelet as claimed in claim 2, wherein the wireless charging module further comprises a rectifier circuit, a voltage control circuit and a charging circuit, the rectifier circuit rectifies the current through the rectifier circuit, the voltage control circuit connects to the rectifier circuit, the voltage control circuit filters spike voltage of the current through the voltage control circuit and outputs a 5V direct current, the charging circuit transfers the 5V direct current to the rechargeable battery to charge the rechargeable battery.

4. The electronic bracelet as claimed in claim 1, further comprising:
a power monitoring module connecting the rechargeable battery and configured to monitor a level of the rechargeable battery.

5. The electronic bracelet as claimed in claim 4, further comprising:
a screen configured to display the level of the rechargeable battery.

6. The electronic bracelet as claimed in claim 4, further comprising:
a switch controlling whether the wireless charging module connects to the rechargeable battery; and
a control module controlling the switch to be turned on to connect the wireless charging module to the rechargeable battery when the level of the rechargeable battery is less than a first preset value.

7. The electronic bracelet as claimed in claim 6, wherein the control module further controls the switch to be turned off to disconnect the wireless charging module to the rechargeable battery when the level of the rechargeable battery is more than a second preset value.

8. The electronic bracelet as claimed in claim 1, wherein the health information comprises blood pressure of the people.

9. The electronic bracelet as claimed in claim 1, wherein the health information comprises heart of the people.

10. The electronic bracelet as claimed in claim 1, further comprising a screen, the screen display the health information.

11. An electronic bracelet comprising:
a rechargeable battery;
a chip configured to detect health information of a wearer of the electronic bracelet; and
a wireless charging module configured to receive an external energy and convert the external energy to 5V direct current to charge the rechargeable battery.

12. The electronic bracelet as claimed in claim 11, wherein the wireless charging module comprises an induction coil, the external energy is alternating magnetic field, the induction coil senses the alternating magnetic field and generate inductive oscillating current.

13. The electronic bracelet as claimed in claim 12, wherein the wireless charging module further comprises a rectifier circuit, a voltage control circuit and a charging circuit, the rectifier circuit rectifies the current through the rectifier circuit, the voltage control circuit connects to the rectifier circuit, the voltage control circuit filters spike voltage of the current through the voltage control circuit and outputs the 5V direct current, the charging circuit transfers the 5V direct current to the rechargeable battery to charge the rechargeable battery.

14. The electronic bracelet as claimed in claim 11, further comprising:
a power monitoring module connecting the rechargeable battery and configured to monitor a level of the rechargeable battery.

15. The electronic bracelet as claimed in claim 14, further comprising:
a screen configured to display the level of the rechargeable battery.

16. The electronic bracelet as claimed in claim 14, further comprising:
a switch controlling whether the wireless charging module connects to the rechargeable battery; and
a control module controlling the switch to be turned on to connect the wireless charging module to the rechargeable battery when the level of the rechargeable battery is less than a first preset value.

17. The electronic bracelet as claimed in claim 16, wherein the control module further controls the switch to be turned off to disconnect the wireless charging module to the rechargeable battery when the level of the rechargeable battery is more than a second preset value.

18. The electronic bracelet as claimed in claim 11, wherein the health information comprises blood pressure of the people.

19. The electronic bracelet as claimed in claim 11, wherein the health information comprises heart of the people.

20. The electronic bracelet as claimed in claim 11, further comprising a screen, the screen display the health information.
